(19) United States
(12) Patent Application Publication
(10) Pub. No.: US 2019/0030366 A1
(43) Pub. Date: Jan. 31, 2019

(54) SYSTEMS AND METHODS FOR RADIOTHERAPY USING ELECTRICAL IMPEDANCE TOMOGRAPHY WITH OTHER IMAGING SYSTEMS

(71) Applicant: UIH-RT US LLC, Concord, CA (US)

(72) Inventor: Jonathan MALTZ, Houston, TX (US)

(73) Assignee: Uih America, Inc., Houston, TX (US)

(21) Appl. No.: 15/663,859

(22) Filed: Jul. 31, 2017

(51) Int. Cl. A61N 5/10; A61B 5/053; A61B 5/00

(52) U.S. Cl. CPC A61N 5/107; A61N 5/1028; A61B 5/0536; A61N 5/1049; A61B 2562/0209; A61N 5/1078; A61N 5/1081; A61N 2005/1074; A61B 5/0037

(57) ABSTRACT

A method for radiotherapy may include generating an electrical impedance tomography (EIT) image of a patient. The method may also include generating a first image. The method may further include determining an EIT feature of the EIT image. The method may also include determining a position relationship between the EIT image and the first image. The method may further include locating an anatomical structure of interest (ASI) of the patient based on the position relationship. The method may further include delivering radiation to the ASI of the patient.

### TECHNICAL FIELD

The present disclosure generally relates to systems and methods for radiotherapy, and more specifically, relates to systems and methods for radiotherapy using electrical impedance tomography (EIT) with other imaging systems.

### BACKGROUND

Radiotherapy is a tumor treatment method by directing ionizing radiation towards the tumor. The radiation may kill the tumor cells as well as healthy human cells nearby. In addition, the tumor and the human organs may be in motion due to physiological activities (for example, breathing, heart beating, blood flowing, muscle contracting and relaxing). Thus, it is desired to track the organ motions and/or the tumor motions during the radiotherapy to precisely deliver the radiation to the tumor and spare the healthy human cells from being radiated.

### SUMMARY

In a first aspect of the present disclosure, a radiotherapy system implemented on one machine and including at least one processor and a storage is provided. The system may include an electrical impedance tomography (EIT) module configured to generate an EIT image of a patient; a first imaging module configured to generate a first image of the patient; a feature determination module configured to determine an EIT feature of the EIT image; a relationship determination module configured to determine a position relationship between the EIT image and the first image; an anatomical structure of interest (ASI) determination module configured to locate an anatomical structure of interest (ASI) of the patient based on the position relationship; and a treatment module configured to deliver radiation to the ASI of the patient.

In some embodiments, the EIT image of the patient may be generated by a plurality of EIT electrodes connected to the patient's body.

In some embodiments, the first image of the patient may include the plurality of EIT electrodes.

In some embodiments, the EIT module may be further configured to reconstruct the EIT image of the patient based on the plurality of EIT electrodes and information contained in the first image.

In some embodiments, the EIT module may be further configured to generate the EIT image of the patient based on information related to positions of the plurality of EIT electrodes obtained from the first image.

In some embodiments, the EIT feature may indicate an observable structure of the EIT image.

In some embodiments, the EIT image may further include a scanned EIT image and a treatment EIT image.

In some embodiments, the relationship determination module may be further configured to generate the position relationship between the EIT image and the first image based on the EIT feature of the scanned EIT image and the ASI in the first image.

In some embodiments, the relationship determination module may be further configured to determine a change in the position relationship during the delivery of the radiation to the ASI of the patient. The treatment module may be further configured to suspend the delivery of the radiation in response to a determination that the change in the position relationship exceeds a pre-set threshold. The system may further include a position adjustment module configured to adjust the position of the patient. The treatment module may be further configured to resume the delivery of the radiation to the ASI of the patient in accordance to the adjustment of the position of the patient.

In another aspect of the present disclosure, a radiotherapy method implemented on one machine and including at least one processor and a storage is provided. The method may include generating an electrical impedance tomography (EIT) image of a patient; generating a first image of the patient; determining an EIT feature of the EIT image; determining a position relationship between the EIT image and the first image; locating an anatomical structure of interest (ASI) of the patient based on the position relationship; and delivering radiation to the ASI of the patient.

In some embodiments, the EIT image of the patient may be generated by a plurality of EIT electrodes connected to the patient's body.

In some embodiments, the first image of the patient may include the plurality of EIT electrodes.

In some embodiments, the method may further include reconstructing the EIT image of the patient based on the plurality of EIT electrodes and information contained in the first image.

In some embodiments, the method may further include generating the EIT image of the patient based on information related to positions of the plurality of EIT electrodes obtained from the first image.

In some embodiments, the EIT feature may indicate an observable structure of the EIT image.

In some embodiments, the EIT image may further include a scanned EIT image and a treatment EIT image.

In some embodiments, the method may further include generating the position relationship between the EIT image and the first image based on the observable structure of the scanned EIT and the ASI in the first image.

In some embodiments, the method may further include determining a change in the position relationship during the delivery of the radiation to the ASI of the patient. The method may further include suspending the delivery of the radiation in response to a determination that the change in the position relationship exceeds a pre-set threshold. The method may also include adjust the position of the patient. The method may further include resuming the delivery of the radiation to the ASI of the patient in accordance to the adjustment of the position of the patient.

In yet another aspect of the present disclosure, a method for radiotherapy is provided. The method may include: generating a scanned electrical impedance tomography (EIT) image of the patient at a first position; generating a first image of the patient; determining an EIT feature of the scanned EIT image; determining an anatomical structure of interest (ASI) in the first image; determining a position relationship between the EIT feature and the ASI based on the scanned EIT image and the first image; generating a treatment EIT image of the patient at a second position; identifying the EIT feature on the treatment EIT image; determining a position of the ASI based on the EIT feature and the position relationship between the EIT feature and the ASI; and delivering radiation to the ASI based on the position of the ASI.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIGS. 1A and 1B are schematic diagrams illustrating an exemplary medical system according to some embodiments of the present disclosure;

FIG. 1C is a schematic diagram illustrating an exemplary medical system with respect to an electrical impedance tomography (EIT) system according to some embodiments of the present disclosure;

FIG. 2 is a schematic diagram illustrating exemplary hardware and/or software components of an exemplary computing device according to some embodiments of the present disclosure;

FIG. 3 is a schematic diagram illustrating exemplary hardware and/or software components of an exemplary mobile device according to some embodiments of the present disclosure;

FIG. 4 is a schematic diagram illustrating an exemplary radiotherapy system according to some embodiments of the present disclosure;

FIG. 5 is a flowchart illustrating an exemplary process/method for radiotherapy according to some embodiments of the present disclosure;

FIG. 6 is a schematic diagram illustrating an exemplary EIT module according to some embodiments of the present disclosure;

FIG. 7 is a flowchart illustrating an exemplary process/method for generating an EIT image according to some embodiments of the present disclosure;

FIG. 8 is a schematic diagram illustrating an exemplary treatment module according to some embodiments of the present disclosure;

FIG. 9 is a flowchart illustrating an exemplary process/method for controlling radiation delivery according to some embodiments of the present disclosure; and

FIG. 10 is a flowchart illustrating an exemplary process/method for performing a radiotherapy operation by using a medical system according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “unit,” “module,” and/or “block” used herein are one method to distinguish different components, elements, parts, section or assembly of different level in ascending order. However, the terms may be displaced by other expression if they achieve the same purpose.

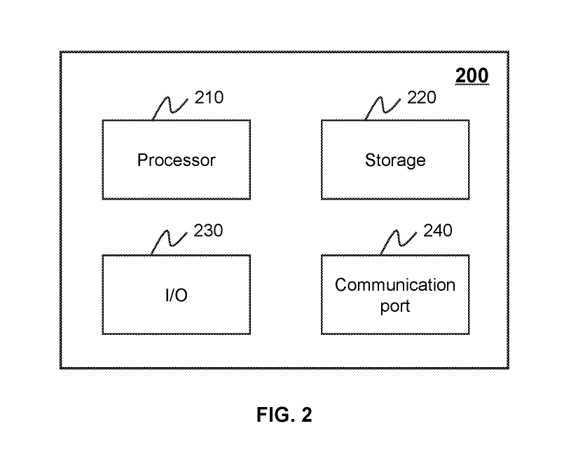

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or other storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices (e.g., processor 210 as illustrated in FIG. 2) may be provided on a computer readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in a firmware, such as an EPROM. It will be further appreciated that hardware modules/units/blocks may be included of connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage.

It will be understood that when a unit, engine, module or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

An aspect of the present disclosure relates to systems and methods for tracking the motions of human anatomical structure during radiotherapy. The present disclosure intends to precisely deliver the radiation to a tumor and spare the healthy organs that are at risk of radiation damage based on the motions of organs during the radiotherapy. By tracking the motion of anatomical structure of interest, the radiation may be delivered more accurately to the tumor, while sparing organs-at-risk (OAR) from the radiation damage.

The term “anatomical structure” in the present disclosure may refer to gas in the patient (e.g., air), liquid in the patient (e.g., water), solid in the patient (e.g., stone), cell of the patient, tissue of the patient, organ of the patient, or any combination thereof, which displayed in medical image (e.g., the EIT image, the first image, etc.), or really existing in or on the patient's body.

The term “location” in the present disclosure may refer to the location of an anatomical structure showing in the medical image, or actual location of the anatomical structure existing in or on the patient body, since medical image may indicate the actual location of a certain anatomical structure existing in or on the patient body.

The term “anatomical structure of interest” (ASI) in the present disclosure may refer to a certain anatomical structure need to be tracked during the radiotherapy. In some embodiments, the ASI may need to be treated by the radiation. In some embodiments, the ASI may be a cell, a tissue, an organ, or any combination thereof. In some embodiments, the ASI may be a tumor, or an organ with tumor, or a tissue with tumor. The term “organ-at-risk” (OAR) in the present disclosure may refer to a cell, an organ or a tissue that close to the ASI and under the risk of radiation damage.

In some embodiments, the electrical impedance tomography (EIT) may track the ASI during the delivery of the radiation. However, owing to poor spatial resolution, the ASI per se may be not observable in EIT image. Thus, location of the ASI may be determined by the EIT and a first imaging system. For example, the ASI may be located and tracked according to a position relationship between an EIT image and a first image, and the motion of an EIT feature of the EIT image.

In some embodiments, the first imaging system may be a computed tomography (CT) system, a magnetic resonance imaging (MRI) system, a positron emission tomography (PET) system, a single photon emission computed tomography (SPECT) system, an ultrasonography system, or the like, or any combination thereof. The EIT image may be reconstructed based on the first image and the information related to the positions of a plurality of EIT electrodes.

The following description is provided to help better understanding methods and/or systems for radiotherapy. The term “image” used in this disclosure may refer to a two dimensional (2D) image, a three dimensional image (3D) image, a four dimensional image (4D) image, or any related image data (e.g., CT data, projection data corresponding to the CT data). This is not intended to limit the scope the present disclosure. For persons having ordinary skills in the art, a certain amount of variations, changes, and/or modifications may be deducted under guidance of the present disclosure. Those variations, changes, and/or modifications do not depart from the scope of the present disclosure.

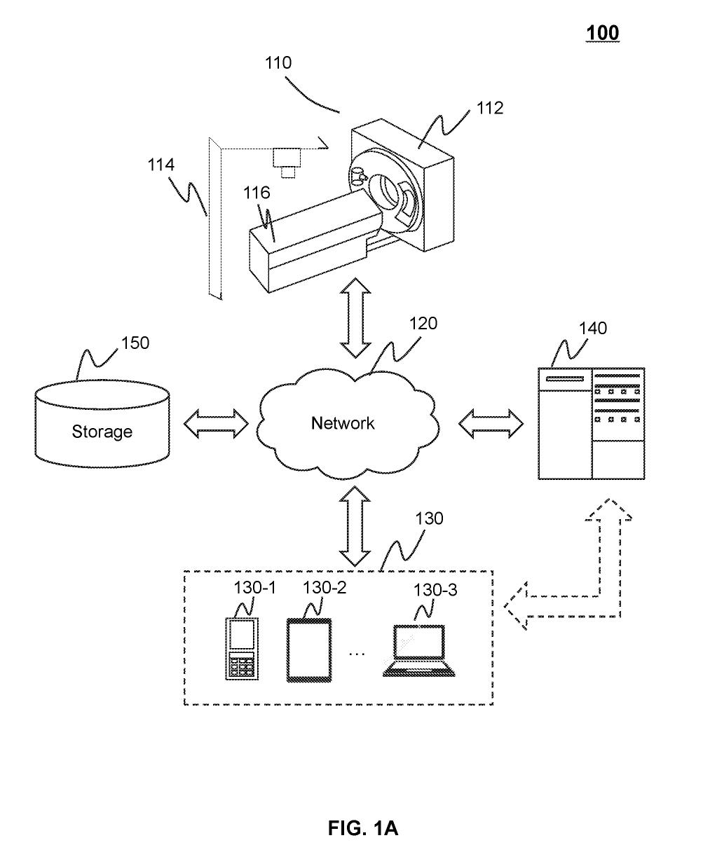

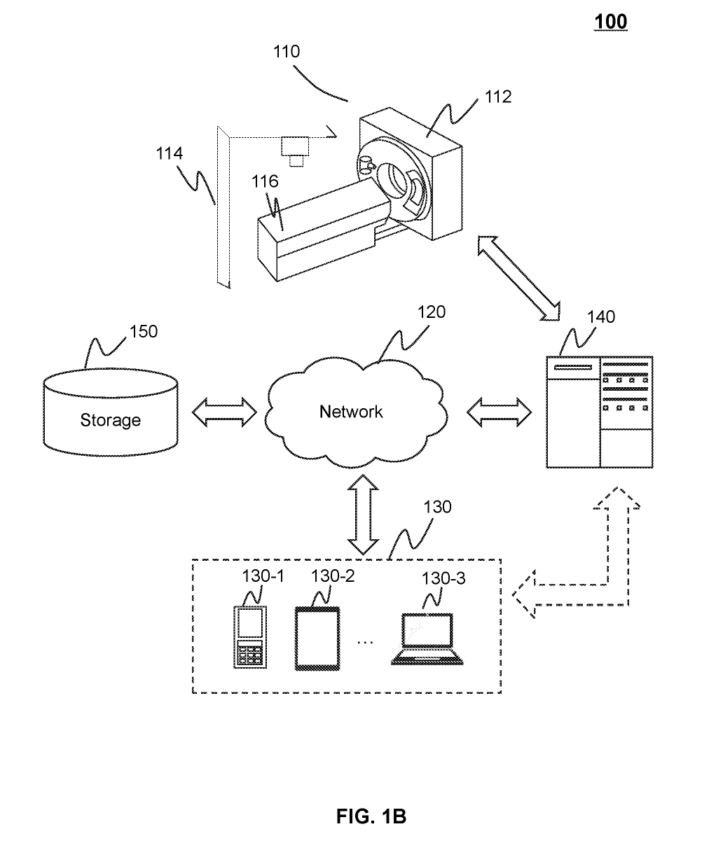

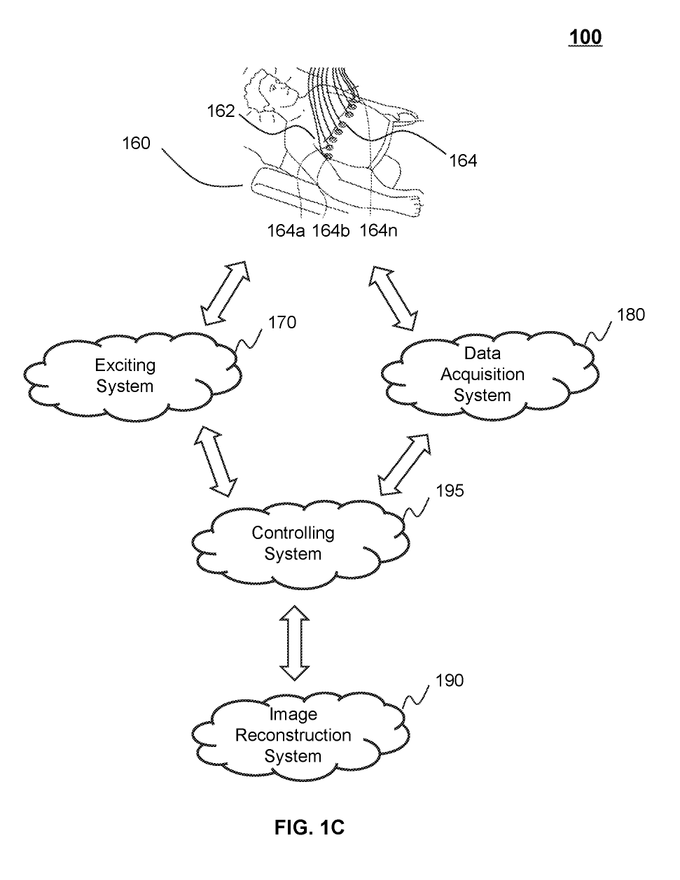

FIGS. 1A and 1B are schematic diagrams illustrating an exemplary medical system 100 according to some embodiments of the present disclosure. The medical system 100 may include a medical device 110, a network 120, a terminal 130, a processing engine 140, and a storage 150.

The medical device 110 may include an imaging machine 112, a treatment machine 114, and a subject couch 116. The imaging machine 112 may be a computed tomography (CT) machine, a magnetic resonance imaging (MRI) machine, a positron emission tomography (PET) machine, a single photon emission computed tomography (SPECT) machine, an ultrasonography machine, or the like, or any combination thereof. As shown in FIGS. 1A and 1B, the imaging machine 112 may include a gantry, an imaging radiation source, a detector, etc. The gantry may support the detector and the imaging radiation source. The treatment machine 114 may include a gantry, a treatment radiation source. The gantry may support the treatment radiation source. A patient may be placed on the subject couch 116. In some embodiments, the imaging machine 112 and a treatment machine 114 may have collinear bores. A patient may be moved from the imaging machine 112 to the treatment machine 114 by transporting the subject couch 116 along the axis of the gantry of the imaging machine 112. In some embodiments, the imaging machine 112 and the treatment machine 114 may have collinear rotation axis. In some embodiments, the imaging machine 112 and the treatment machine 114 may be integrated to a medical device (not shown in FIGS. 1A and 1B). For example, the imaging machine 112 and the treatment machine 114 may share a same radiation source. As another example, the radiation source for treatment and the radiation source for imaging may be mounted on a same gantry.

The network 120 may facilitate exchange of information and/or data. In some embodiments, one or more components in the medical system 100 (e.g., the medical device 110, the terminal 130, the processing engine 140, or the storage 150) may send information and/or data to other component(s) in the medical system 100 via the network 120. For example, the processing engine 140 may obtain image data from the medical device 110 via the network 120. As another example, the processing engine 140 may obtain user instructions from the terminal 130 via the network 120. In some embodiments, the network 120 may be any type of wired or wireless network, or combination thereof. Merely by way of example, the network 120 may include a cable network, a wireline network, an optical fiber network, a tele communications network, an intranet, an Internet, a local area network (LAN), a wide area network (WAN), a wireless local area network (WLAN), a metropolitan area network (MAN), a wide area network (WAN), a public telephone switched network (PSTN), a Bluetooth network, a ZigBee network, a near field communication (NFC) network, or the like, or any combination thereof. In some embodiments, the network 120 may include one or more network access points. For example, the network 120 may include wired or wireless network access points such as base stations and/or internet exchange points through which one or more components of the CT system 100 may be connected to the network 120 to exchange data and/or information.

The terminal 130 includes a mobile device 130-1, a tablet computer 130-2, a laptop computer 130-3, or the like, or any combination thereof. In some embodiments, the mobile device 130-1 may include a smart home device, a wearable device, a smart mobile device, a virtual reality device, an augmented reality device, or the like, or any combination thereof. In some embodiments, the smart home device may include a smart lighting device, a control device of an intelligent electrical apparatus, a smart monitoring device, a smart television, a smart video camera, an interphone, or the like, or any combination thereof. In some embodiments, the wearable device may include a smart bracelet, a smart footgear, a smart glass, a smart helmet, a smart watch, a smart clothing, a smart backpack, a smart accessory, or the like, or any combination thereof. In some embodiments, the smart mobile device may include a smartphone, a personal digital assistance (PDA), a gaming device, a navigation device, a point of sale (POS) device, or the like, or any combination thereof. In some embodiments, the virtual reality device and/or the augmented reality device may include a virtual reality helmet, a virtual reality glass, a virtual reality patch, an augmented reality helmet, an augmented reality glass, an augmented reality patch, or the like, or any combination thereof. For example, the virtual reality device and/or the augmented reality device may include a Google Glass, an Oculus Rift, a Hololens, a Gear VR, etc. The terminal 130 may remotely operate the imaging machine 112 or the treatment machine 114. In some embodiments, the terminal 130 may operate the imaging machine 112 or the treatment machine 114 via a wireless connection. In some embodiments, the terminal 130 may receive information and/or instructions inputted by a user, and transmit the received information and/or instructions to the imaging machine 112 or the treatment machine 114 or to the processing engine 140 via the network 120. In some embodiments, the terminal 130 may receive data and/or information from the processing engine 140. In some embodiments, the terminal 130 may be part of the processing engine 140. In some embodiments, the terminal 130 may be omitted.

The processing engine 140 may process data and/or information obtained from the medical device 110, the terminal 130, or the storage 150. For example, the processing engine 140 may process image data and determine a regularization item that may be used to modify the image data. In some embodiments, the processing engine 140 may be a single server, or a server group. The server group may be centralized, or distributed. In some embodiments, the processing engine 140 may be local or remote. For example, the processing engine 140 may access information and/or data stored in the medical device 110, the terminal 130, and/or the storage 150 via the network 120. As another example, the processing engine 140 may be directly connected to the medical device 110, the terminal 130 and/or the storage 150 to access stored information and/or data. In some embodiments, the processing engine 140 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof. In some embodiments, the processing engine 140 may be implemented on a computing device 200 having one or more components illustrated in FIG. 2 in the present disclosure.

The storage 150 may store data and/or instructions. In some embodiments, the storage 150 may store data obtained from the terminal 130 and/or the processing engine 140. In some embodiments, the storage 150 may store data and/or instructions that the processing engine 140 may execute or use to perform exemplary methods described in the present disclosure. In some embodiments, the storage 150 may include a mass storage, a removable storage, a volatile read-and-write memory, a read-only memory (ROM), or the like, or any combination thereof. Exemplary mass storage may include a magnetic disk, an optical disk, a solid-state drive, etc. Exemplary removable storage may include a flash drive, a floppy disk, an optical disk, a memory card, a zip disk, a magnetic tape, etc. Exemplary volatile read-and-write memory may include a random access memory (RAM). Exemplary RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. Exemplary ROM may include a mask ROM (MROM), a programmable ROM (PROM), an erasable programmable ROM (PEROM), an electrically erasable programmable ROM (EEPROM), a compact disk ROM (CD-ROM), and a digital versatile disk ROM, etc. In some embodiments, the storage 150 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof.

In some embodiments, the storage 150 may be connected to the network 120 to communicate with one or more components in the medical system 100 (e.g., the processing engine 140, the terminal 130). One or more components in the medical system 100 may access the data or instructions stored in the storage 150 via the network 120. In some embodiments, the storage 150 may be directly connected to or communicate with one or more components in the medical system 100 (e.g., the processing engine 140, the terminal 130). In some embodiments, the storage 150 may be part of the processing engine 140.

FIG. 1C is a schematic diagram illustrating an exemplary medical system 100 with respect to an electrical impedance tomography (EIT) system according to some embodiments of the present disclosure. The EIT system may include an EIT device 160, an exciting system 170, a data acquisition system 180, an image reconstruction system 190, and a controlling system 195.

The EIT device 160 may include a plurality of electrodes 164 (e.g., 164a, 164b . . . 164n) placing on the skin of a patient's body 162, shown in FIG. 1C. In some embodiments, the plurality of electrodes 164 may be placed within body cavities or orifices of the patient. The plurality of electrodes may be fabricated from a low density material with high atomic number relative to that of most human body tissues, e.g., calcium. The advantage of using such a material for the EIT electrodes is that such materials may be imaged with high contrast in X-ray images, owing to relatively high atomic number relative to human body tissues such as skin. This may improve the visibility of the electrodes in CT images. If the atomic number of the material is too high (e.g. gold), this may introduce artifacts in the CT images, which would compromise the quality of the CT images. Low density material is preferred in order to reduce the effect of the presence of the electrode on the treatment radiation incident on or near the electrode. The plurality of electrodes may be identified on an image generated by the imaging machine 112.

The exciting system 170 may impose a current or voltage on the patient's body 162 via the plurality of electrodes 164. The exciting system 170 may implement a current exciting method, a voltage exciting method, an induced current exciting method, or the like, or a combination thereof.

The data acquisition system 180 may collect electrical impedance data (such as the conductivity, permittivity, and impedance) relating to anatomical structure of the patient's body 162 via the plurality of electrodes 164. In some embodiments, the collected electrical impedance information and/or data may be stored in the data acquisition system 180. In some embodiments, the data acquisition system 180 may be connected to the network 120 to communicate with one or more components in the system 100 (e.g., the storage 150, the EIT device 160, or the image reconstruction system 190). For example, the collected electrical impedance information and/or data by the data acquisition system 180 may be sent to the storage 150 via the network 120. In some embodiments, the data acquisition system 180 may be directly connected to or communicate with one or more components in the system 100 (e.g., the storage 150, the EIT device 160, or the image reconstruction system 190). In some embodiments, the data acquisition system 180 may be part of the processing engine 140.

The image reconstruction system 190 may access the electrical impedance data stored in the data acquisition system 180 and/or the storage 150 via the network 120. In some embodiments, the image reconstruction system 190 may be directly connected to or communicate with the data acquisition system 180 and/or the storage 150. In some embodiments, the image reconstruction system 190 may be part of the processing engine 140. The image reconstruction system 190 may reconstruction the EIT image using a reconstruction algorithm based on a finite element theory.

The controlling system 195 may be a single server, or a server group. The server group may be centralized, or distributed. In some embodiments, the controlling system 195 may be local or remote. For example, the controlling system 195 may control the exciting system 170, the data acquisition system 180, and/or the image reconstruction system 190 via the network 120. As another example, the controlling system 195 may be directly connected to the exciting system 170, the data acquisition system 180, and/or the image reconstruction system 190. In some embodiments, the controlling system 195 may be implemented on a computing device 200 having one or more components illustrated in FIG. 2 in the present disclosure.

FIG. 2 is a schematic diagram illustrating exemplary hardware and/or software components of an exemplary computing device 200 on which the processing engine 140 may be implemented according to some embodiments of the present disclosure. As illustrated in FIG. 2, the computing device 200 may include a processor 210, a storage 220, an input/output (I/O) 230, and a communication port 240.

The processor 210 may execute computer instructions (program code) and perform functions of the processing engine 140 in accordance with techniques described herein. The computer instructions may include routines, programs, objects, components, data structures, procedures, modules, and functions, which perform particular functions described herein. For example, the processor 210 may process image data obtained from the imaging machine 112, the terminal 130, the storage 150, or any other component of the medical system 100. In some embodiments, the processor 210 may include a microcontroller, a microprocessor, a reduced instruction set computer (RISC), an application specific integrated circuits (ASICs), an application-specific instruction-set processor (ASIP), a central processing unit (CPU), a graphics processing unit (GPU), a physics processing unit (PPU), a microcontroller unit, a digital signal processor (DSP), a field programmable gate array (FPGA), an advanced RISC machine (ARM), a programmable logic device (PLD), any circuit or processor capable of executing one or more functions, or the like, or any combinations thereof.

Merely for illustration, only one processor is described in the computing device 200. However, it should be note that the computing device 200 in the present disclosure may also include multiple processors, thus operations and/or method steps that are performed by one processor as described in the present disclosure may also be jointly or separately performed by the multiple processors. For example, if in the present disclosure the processor of the computing device 200 executes both step A and step B, it should be understood that step A and step B may also be performed by two different processors jointly or separately in the computing device 200 (e.g., a first processor executes step A and a second processor executes step B, or the first and second processors jointly execute steps A and B).

The storage 220 may store data/information obtained from the imaging machine 112, the terminal 130, the storage 150, or any other component of the medical system 100. In some embodiments, the storage 220 may include a mass storage, a removable storage, a volatile read-and-write memory, a read-only memory (ROM), or the like, or any combination thereof. For example, the mass storage may include a magnetic disk, an optical disk, a solid-state drive, etc. The removable storage may include a flash drive, a floppy disk, an optical disk, a memory card, a zip disk, a magnetic tape, etc. The volatile read-and-write memory may include a random access memory (RAM). The RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. The ROM may include a mask ROM (MROM), a programmable ROM (PROM), an erasable programmable ROM (PEROM), an electrically erasable programmable ROM (EEPROM), a compact disk ROM (CD-ROM), and a digital versatile disk ROM, etc. In some embodiments, the storage 220 may store one or more programs and/or instructions to perform exemplary methods described in the present disclosure. For example, the storage 220 may store a program for the processing engine 140 for determining a regularization item.

The I/O 230 may input or output signals, data, or information. In some embodiments, the I/O 230 may enable a user interaction with the processing engine 140. In some embodiments, the I/O 230 may include an input device and an output device. Exemplary input device may include a keyboard, a mouse, a touch screen, a microphone, or the like, or a combination thereof. Exemplary output device may include a display device, a loudspeaker, a printer, a projector, or the like, or a combination thereof. Exemplary display device may include a liquid crystal display (LCD), a light-emitting diode (LED)-based display, a flat panel display, a curved screen, a television device, a cathode ray tube (CRT), or the like, or a combination thereof.

The communication port 240 may be connected to a network (e.g., the network 120) to facilitate data communications. The communication port 240 may establish connections between the processing engine 140 and the medical device 110, the terminal 130, or the storage 150. The connection may be a wired connection, a wireless connection, or combination of both that enables data transmission and reception. The wired connection may include electrical cable, optical cable, telephone wire, or the like, or any combination thereof. The wireless connection may include Bluetooth, Wi-Fi, WiMax, WLAN, ZigBee, mobile network (e.g., 3G, 4G, 5G, etc.), or the like, or a combination thereof. In some embodiments, the communication port 240 may be a standardized communication port, such as RS232, RS485, etc. In some embodiments, the communication port 240 may be a specially designed communication port. For example, the communication port 240 may be designed in accordance with the digital imaging and communications in medicine (DICOM) protocol.

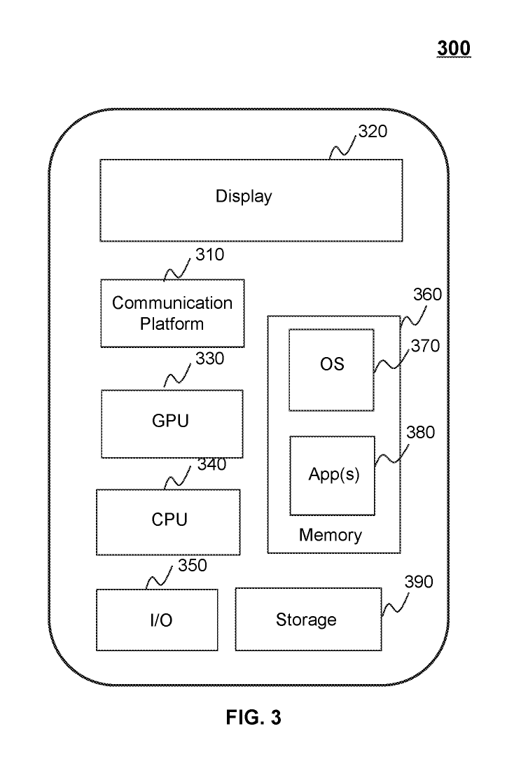

FIG. 3 is a schematic diagram illustrating exemplary hardware and/or software components of an exemplary mobile device 300 on which the terminal 130 may be implemented according to some embodiments of the present disclosure. As illustrated in FIG. 3, the mobile device 300 may include a communication platform 310, a display 320, a graphic processing unit (GPU) 330, a central processing unit (CPU) 340, an I/O 350, a memory 360, and a storage 390. In some embodiments, any other suitable component, including but not limited to a system bus or a controller (not shown), may also be included in the mobile device 300. In some embodiments, a mobile operating system 370 (e.g., iOS, Android, Windows Phone, etc.) and one or more applications 380 may be loaded into the memory 360 from the storage 390 in order to be executed by the CPU 340. The applications 380 may include a browser or any other suitable mobile apps for receiving and rendering information relating to image processing or other information from the processing engine 140. User interactions with the information stream may be achieved via the I/O 350 and provided to the processing engine 140 and/or other components of the medical system 100 via the network 120.

To implement various modules, units, and their functionalities described in the present disclosure, computer hardware platforms may be used as the hardware platform(s) for one or more of the elements described herein. The hardware elements, operating systems and programming languages of such computers are conventional in nature, and it is presumed that those skilled in the art are adequately familiar therewith to adapt those technologies to the tracking of the motions of human anatomical structure during radiotherapy as described herein. A computer with user interface elements may be used to implement a personal computer (PC) or other type of work station or terminal device, although a computer may also act as a server if appropriately programmed. It is believed that those skilled in the art are familiar with the structure, programming and general operation of such computer equipment and as a result the drawings should be self-explanatory.

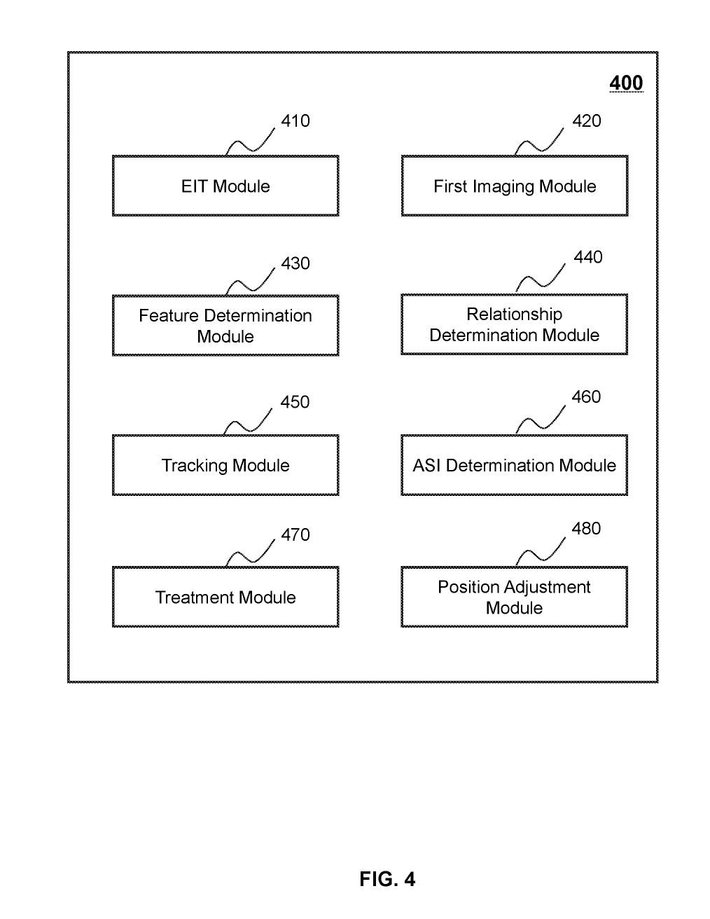

FIG. 4 is a schematic diagram illustrating an exemplary radiotherapy system 400 according to some embodiments of the present disclosure. The radiotherapy system 400 may perform operations including, for example, electrical impedance tomography (EIT) image generation, first image generation, EIT feature determination, or the like, or any combination thereof. The radiotherapy system 400 may include an EIT module 410, a first imaging module 420, a feature determination module 430, a relationship determination module 440, a tracking module 450, an anatomical structure of interest (ASI) determination module 460, a treatment module 470, and a position adjustment module 480.

The EIT module 410 may be configured to generate an EIT image. In some embodiments, the EIT image may be generated according to electrical impedance data (such as the conductivity, permittivity, and impedance) relating to anatomical structure of the patient's body. The anatomical structure of the patient may be identified in the EIT image accordingly. The electrical impedance data associated with an anatomical structure of the patient may be acquired by a plurality of EIT electrodes connected to the patient's body. In some embodiments, the electrical impedance data associated with the anatomical structure of the patient may be acquired by the plurality of EIT electrodes placed on the skin of the patient's body. In some embodiments, the electrical impedance data associated with the anatomical structure of the patient may be acquired by the plurality of EIT electrodes implanted in the patient, for example, placed within body cavities or orifices of the patient. The EIT image may be generated according to the electrical impedance data and information relating to the plurality of EIT electrodes.

In some embodiments, the EIT image may include scanned EIT image and treatment EIT image. The scanned EIT image may be generated before the radiotherapy. The treatment EIT image may be continuously generated during the radiotherapy, and thus, the motions of an anatomical structure of interest (ASI) may be tracked. To avoid potential interference between the EIT device 160 and pulsed radiation sources such as linear accelerators (linac), the excitation of the EIT device 160 may be timed. For example, the exciting system 170 may cease excitation of the EIT device 160, and/or the data acquisition system 180 may cease acquiring of the EIT signal and/or data while the linac pulses are delivered such that interference is avoided or minimized. Since the duty cycle of a pulsed linac is of the order of 1/1000 (on time/off time), quality of the EIT image should not be compromised according to this scheme. Equivalently, the acquired EIT signal and/or data may be marked with timestamps corresponding to the linac pulses, and those parts of the record marked has having been recorded during the pulses discarded or suitably filtered in the image reconstruction process.

The first imaging module 420 may be configured to generate a first image of the patient. The first image may be a medical image, for example, a CT image, of which the spatial resolution is higher than that of the EIT image. Thus, the anatomical structure of interest of the patient may be clearly displayed in the first image.

In some embodiments, the first image of the patient may be generated before the radiotherapy. In some embodiments, the first image and the scanned EIT image of the patient may be generated simultaneously with the plurality of EIT electrodes connected to the patient's skin, for example, placed on the skin of the patient's body or within body cavities or orifices of the patient. Thus, the first image may include information relating to the plurality of EIT electrodes, and the EIT module 410 may use the information relating to the plurality of EIT electrodes to reconstruct the EIT image for a higher resolution. In some embodiments, the EIT module 410 may also reconstruct the EIT image according to information contained in the first image.

The feature determination module 430 may be configured to determine an EIT feature of an EIT image. In some embodiments, the EIT feature of the EIT image may indicate a certain anatomical structure of the patient. Even the tumor may not be observable in the EIT image, some other feature may be observable and can be identified in the EIT image, such as a certain organ showing in the EIT image and the first image (e.g., diaphragm, liver, heart, etc.), a certain anatomical structure showing in the EIT image and the first image (e.g., skin, membrane, etc.), or a shape representing certain anatomical structure (e.g., curve, circle in the EIT image). In some embodiments, the electrical impedance data in the EIT image may vary with different anatomical structures. If electrical impedance of an anatomical structure is larger than a threshold, the anatomical structure may be identified in the EIT image. The anatomical structure may be determined as a feature of the EIT image. The word “observable” may refer to that the structure or the feature (e.g., diaphragm, liver, skin, etc.) can be seen by naked eyes of human beings in the EIT image (e.g., larger than 0.55 mm in the EIT image). In some embodiments, the feature determination module 430 may determine the same EIT feature in the scanned EIT image and the treatment EIT image. In some embodiments, the feature determination module 430 may determine the same EIT feature in the first image and the EIT image. Since the EIT feature represents a certain anatomical structure of the patient, the EIT feature existing in the EIT image may also exist in the first image. In some embodiments, the EIT feature of the EIT image may serve as a suitable surrogate for the ASI.

In some embodiments, generation of the first image and the scanned EIT image may occur before the radiation treatment. During the generation of the first image and the scanned EIT image, the ASI of the patient may be in motion. When the ASI is in a first motion state (e.g., the diaphragm moves to a certain position, or cardiac motion is in a certain state), the EIT module 410 may generate a scanned EIT image M1 of the patient corresponding to the first motion state, and the first imaging module 420 may generate a first image N1 of the patient corresponding to the first motion state. Similarly, the EIT module 410 and the first imaging module 420 may generate a scanned EIT image M2 and a first image N2 corresponding to a second motion state of the organ. Thus, the EIT module 410 may generate several scanned EIT images and the first imaging module 420 may generate several first images corresponding to different motion states.

The relationship determination module 440 may be configured to determine a position relationship between an EIT image and a first image. In some embodiments, the position relationship may associate a position of an ASI (such as the tumor) in the first image with a position of the EIT feature in the scanned EIT image.

Based on the several first images (such as N1, N2, etc.) and several scanned EIT images (such as M1, M2, etc.), the relationship determination module 440 may determine the precise location of the ASI (such as the tumor) showing in the several first images. For example, the relationship determination module 440 may generate location data y₁ related to the ASI in first image N1, and generate location data y₂ related to the ASI in first image N2, etc. The relationship determination module 440 may generate location data x₁ related to the EIT feature in the scanned EIT image M1, and generate location data x₂ related to the EIT feature in the scanned EIT image M2. Thus, the relationship determination module 440 may generate a position relationship y=f(x) to denote a position relationship between an EIT image and a first image based on the location data related to the EIT feature (such as x₁, x₂, etc.) and location data related to the ASI (such as y₁, y₂, etc.), wherein y may denote a position of the anatomical structure in the first image and x may denote a position of the EIT feature in the scanned EIT image. Position of the ASI may be determined based on the position relationship y=f(x) and a known location of the EIT feature. In some embodiments, the known location of the EIT feature may be determined in the treatment EIT image.

The tracking module 450 may be configured to track the motions of an EIT feature of an EIT image. In some embodiments, the tracking module 450 may track the EIT feature of the treatment EIT image. In some embodiments, the tracking module 450 may track the motions of the EIT feature at time intervals, such as every 20 milliseconds, 50 milliseconds, 100 milliseconds, etc. The tracking module 450 may also determine one or more movement trends of the EIT feature. The current location of the EIT feature may be determined according to a previous location and the one or more movement trends of the EIT feature. Since the treatment EIT image is continuously generated during the radiotherapy, the tracking module 450 may continuously track the motions of the EIT feature and determine a plurality of locations of the EIT feature in the treatment EIT image in real time. In some embodiments, the tracking module 450 may determine one or more movement patterns of an ASI. The movement patterns may include a movement pattern associated with the breathing motion of the patient, a movement pattern associated with the cardiac motion of the patient, etc. The tracking module 450 may determine the one or more movement patterns of the ASI according to the position relationship y=f(x) and the plurality of locations of the corresponding EIT feature. Based on the one or more movement patterns, the tracking module 450 may predict locations of the ASI within a time period during which the radiation will be delivered. The time period may be 50, 100, or 150 milliseconds.

The ASI determination module 460 may be configured to locate the ASI of a patient based on the position relationship. As described elsewhere in the present disclosure, during the radiotherapy, the tracking module 450 may continuously determine the locations of the EIT feature in the treatment EIT image. Based on the known locations of the EIT feature obtained by the tracking module 450 and the position relationship y=f(x) generated by the relationship determination module 440, the ASI determination module 460 may determine the locations of the ASI in real time. Thus, motions of the ASI may be continuously monitored in real time.

The treatment module 470 may be configured to deliver radiation to the ASI. With the locations of the ASI known in real time, the treatment module 470 may accurately deliver the radiation to the ASI despite that the ASI is in motion. In some embodiments, the radiation delivery may be determined according to a predetermined treatment plan, which may include a radiation dose, a radiation time, or the like, or any combination thereof. For example, the treatment module 470 may start the delivery the radiation to the ASI when the position of the ASI is conformed to the predetermined treatment plan. As the location of the ASI can be predicted by the tracking module 450, the treatment module 470 may determine the delivery of the radiation to the ASI by applying a treatment plan that conforms to the position of the ASI. For example, when the tracking module 450 predicts the locations of the ASI during a time period, the treatment module 470 may deliver the radiation to the ASI according to a treatment plan that conforms to the predicted location during the time period.

The position adjustment module 480 may be configured to adjust a position of a patient with respect to an imaging bore or a radiotherapy bore. In some embodiments, before the radiotherapy, the position adjustment module 480 may place the patient on an initial setup position by moving the subject couch 116. The initial setup position may be an isocenter of a medical machine, e.g., the imaging machine 112, or the treatment machine 114. Through the imaging bore, the patient may be scanned for generating the scanned EIT image and the first image, and through the radiotherapy bore, the patient may be radiated and scanned for generating the treatment EIT image. In some embodiments, the imaging machine 112 and the treatment machine 114 may share a same bore. The radiation source in the bore may emit rays with a certain level of energy (e.g., greater than 160 keV) for treatment. In some embodiments, the radiation source in the bore may emit rays with a different level of energy (e.g., generally less than 160 keV) for imaging. By configuring different radiation energy levels for treatment and imaging, respectively, the position adjustment module 480 may not need to adjust the position of the patient from the imaging bore to the radiotherapy bore. In some embodiments, during the radiotherapy, the position adjustment module 480 may adjust the position of the patient with respect to the radiotherapy bore corresponding to a change of the position relationship between the EIT image and the first image. In some embodiments, the position adjustment module 480 may also be configured to adjust radiation area relative to the patient. For example, the position adjustment module 480 may adjust the position of the radiation source relative to the patient, so that the treatment module 470 may deliver the radiation to the ASI and spare the organs-at-risk (OAR). In another example, the position adjustment module 480 may adjust the position of the patient relative to the radiation source, so that the treatment module 470 may deliver the radiation to the ASI and spare the organs-at-risk (OAR). In another example, the position adjustment module 480 may adjust the collimators of the radiation source, so that the treatment module 470 may deliver the radiation to the ASI and spare the organs-at-risk (OAR).

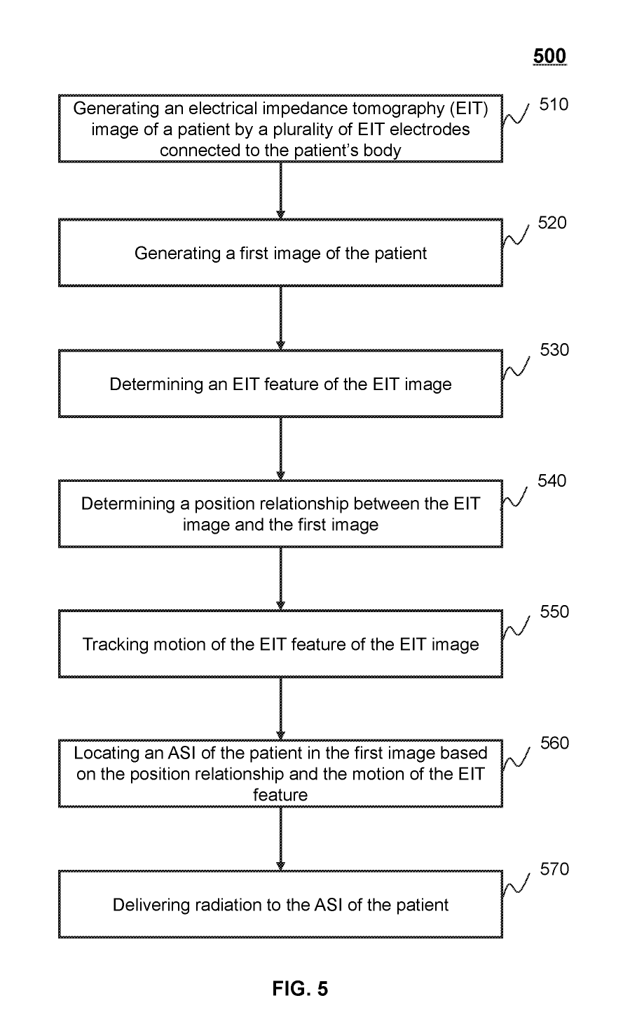

FIG. 5 is a flowchart illustrating an exemplary process/method 500 for radiotherapy according to some embodiments of the present disclosure. The process and/or method 500 may be executed by the medical system 100. For example, the process and/or method 500 may be implemented as a set of instructions (e.g., an application) stored in the storage 220. The processor 210 may execute the set of instructions and may accordingly be directed to perform the process and/or method 500. The operations of the illustrated process/method presented below are intended to be illustrative. In some embodiments, the process/method may be accomplished with one or more additional operations not described, and/or without one or more of the operations discussed. Additionally, the order in which the operations of the process/method as illustrated in FIG. 5 and described below is not intended to be limiting.

Before radiotherapy, the position adjustment module 480 may place a patient on an initial setup position. The medical machine may be the imaging machine 112, or the treatment machine 114. In some embodiments, the patient may be placed on the subject couch 116 to receive radiation. The patient may be placed on the initial setup position by moving the subject couch 116.

In step 510, the EIT module 410 may generate an electrical impedance tomography (EIT) image of a patient by a plurality of EIT electrodes connected to the patient's body. In some embodiments, a plurality of EIT electrodes may be placed on the skin of the patient's body. In some embodiments, the plurality of EIT electrodes may be placed within body cavities or orifices of the patient. The plurality of EIT electrodes may be fabricated from a low density material with high atomic number relative to that of human body tissues, e.g., calcium. The advantage of using such a material for the EIT electrodes is that such materials may be imaged with high contrast in X-ray images, owing to relatively high atomic number relative to tissues such as skin. This may improve the visibility of the electrodes in CT images. If the atomic number of the material is too high (e.g. gold), this may introduce artifacts in the CT images, which would compromise the quality of the CT images. Low density material is preferred in order to reduce the effect of the presence of the electrode on the treatment radiation incident on or near the electrode. In some embodiments, the EIT image may be a reconstructed image including a bone of the patient, a tissue of the patient, an organ of the patient, or the like, or any combination thereof. In some embodiments, the EIT image may be generated according to acquired electrical impedance data, information contained in an image and information relating to the EIT electrodes.

In step 520, the first imaging module 420 may generate a first image of the patient. In some embodiments, the first image may be generated using an imaging system. The imaging system may be a computed tomography (CT) system, a magnetic resonance imaging (MRI) system, a positron emission tomography (PET) system, a single photon emission computed tomography (SPECT) system, an ultrasonography system, or the like, or any combination thereof. In some embodiments, the first image may be a two-dimensional image, a three-dimensional image, a four-dimensional image, etc. The first image of the patient may be obtained to include information related to the positions of the plurality of EIT electrodes by imaging a body part of the patient and the plurality of EIT electrodes simultaneously. Thus, the positions of the plurality of EIT electrodes may be determined according to the first image. It should be noted that steps 510 and 520 should be performed simultaneously. In some embodiments, step 520 may be performed before step 510.

In step 530, the feature determination module 430 may determine an EIT feature of the EIT image. In some embodiments, the feature determination module 430 may determine an EIT feature of the scanned EIT image in step 530. In some embodiments, the EIT feature may indicate an anatomical structure showing in the scanned EIT image. For example, the EIT feature may be an observable structure of the EIT image. The word “observable” may refer to the structure or the feature can be seen by naked eyes of human beings in the EIT image (e.g., larger than 0.55 mm in the EIT image). In some embodiments, the feature of the EIT image may serve as a suitable surrogate for an anatomical structure of interest (ASI).

In step 540, the relationship determination module 440 may determine a position relationship between the EIT image and the first image. In some embodiments, the relationship determination module 440 may determine a position relationship between the scanned EIT image and the first image. For example, the relationship determination module 440 may determine the position relationship between the scanned EIT image and the first image based on the EIT feature of the scanned EIT image and the ASI in the first image. In some embodiments, the position relationship may associate a position of the ASI in the first image with a position of the EIT feature in the scanned EIT image.

In step 550, the tracking module 450 may track the motion of the EIT feature of the EIT image. As the EIT feature may be an observable structure of the EIT image according to the human being's vision perception, the motion of the EIT feature may be observed from the EIT image. The motion of the EIT feature may be tracked continuously. Alternatively or additionally, the motion of the EIT feature may be tracked at time intervals such as every 20 milliseconds, 50 milliseconds, every 100 milliseconds, etc. The tracking module 450 may also determine one or more movement trends of the EIT feature. The current location of the EIT feature may be determined according to a previous location and the one or more movement trends of the EIT feature. Since the treatment EIT image is continuously generated during the radiotherapy, the tracking module 450 may continuously track the motions of the EIT feature and determine a plurality of locations of the EIT feature in the treatment EIT image in real time. In some embodiments, the tracking module 450 may determine one or more movement patterns of an ASI. For example, the movement pattern may include a movement pattern associated with the breathing motion of the patient, a movement pattern associated with the cardiac motion of the patient, etc. The tracking module 450 may determine the one or more movement patterns of the ASI according to the position relationship y=f(x) and the plurality of locations of the corresponding EIT feature. Based on the one or more movement patterns, the tracking module 450 may predict locations of the ASI within a time period during which the radiation will be delivered. The time period may be 50, 100, or 150 milliseconds.

In step 560, the ASI determination module 460 may locate an ASI of the patient in the first image based on the position relationship and the motion of the EIT feature. In some embodiments, the ASI may move due to various motions the patient, for example, cardiac motions of the heart, respiratory motions of the lungs and/or the diaphragm, blood flowing, muscle contracting and relaxing, or the like, or any combination thereof. As the motion of the EIT feature may be tracked on the EIT image dynamically, the motion of the ASI of the patient may be dynamically determined according to the position relationship and the motion of the EIT feature.

In step 570, the treatment module 470 may deliver radiation to the ASI of the patient. In some embodiments, the treatment module 470 may deliver radiation to the ASI of the patient according to a predetermined treatment plan. The predetermined treatment plan may include a radiation dose, a radiation time, or the like, or any combination thereof. For example, the treatment module 470 may start the delivery of the radiation to the ASI when the position of the ASI is conformed to a predetermined treatment plan. As the location of the ASI can be predicted by the tracking module 450, the treatment module 470 may determine the delivery of the radiation to the ASI by applying a treatment plan that conforms to the position of the ASI. For example, the position adjustment module 480 may adjust the radiation source to target at the predicted location of the ASI, and then the treatment module 470 may deliver the radiation to the ASI by applying a treatment plan that conforms to the predicted location of the ASI. In some embodiments, the treatment module 470 may suspend the delivery of the radiation to the ASI if a change in the position relationship between the ETI image and the first image exceeds a pre-set threshold.

It should be noted that the above description of the process/method for radiotherapy is provided for the purposes of illustration, and is not intended to limit the scope of the present disclosure. For persons having ordinary skills in the art, multiple variations and modifications may be made under the teachings of the present disclosure. However, those variations and modifications do not depart from the scope of the present disclosure. In some embodiments, step 550 may be omitted, the position of the ASI may be determined based on the position relationship y=f(x) and known location of the corresponding EIT feature. Then the delivery of the radiation to the ASI may be started when the position of the ASI is conformed to a predetermined treatment plan.

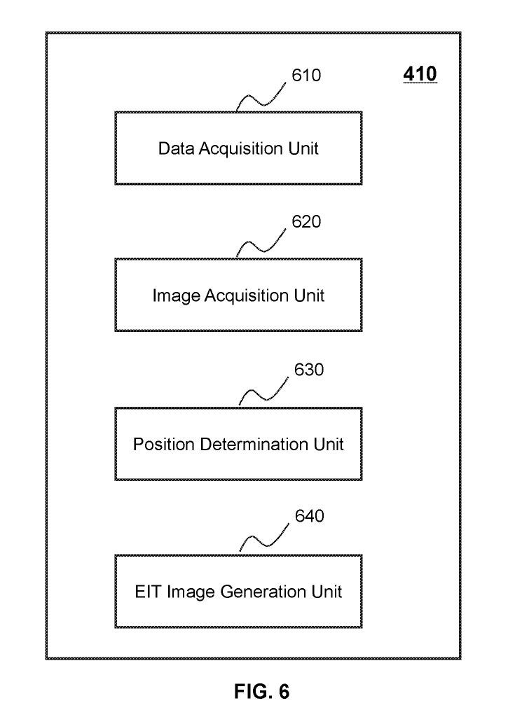

FIG. 6 is a schematic diagram illustrating an exemplary EIT module 410 according to some embodiments of the present disclosure. The EIT module 410 may include a data acquisition unit 610, an image acquisition unit 620, a position determination unit 630, and an EIT image generation unit 640.

The data acquisition unit 610 may be configured to acquire data relating to a patient via a plurality of electrical impedance tomography (EIT) electrodes. In some embodiments, the data may include voltage data, current data, or the like, or any combination thereof. The electrical impedance data may be determined according to the current data, the voltage data, etc.

The image acquisition unit 620 may be configured to acquire a first image of the patient including the plurality of EIT electrodes. The first image of the patient may be obtained to include position information related to the plurality of EIT electrodes by imaging a body part of the patient and the plurality of EIT electrodes simultaneously. In some embodiments, the first image may be used to reconstruct the EIT image. For example, in the first image, there are some anatomical structures of the patient that is static, without motion. Data of the static anatomical structure may be used as constant value, while the EIT image data may be used to reconstruct the anatomical structures in motion. In some embodiments, the first image may include information relating to electrical characteristics of the plurality of EIT electrodes. The electrical characteristics of the plurality of EIT electrodes may be determined from the first image. In some embodiments, the electrical characteristics of the plurality of EIT electrodes may be used to reconstruct the EIT image, and resolution of a reconstructed EIT image may be improved accordingly.

The position determination unit 630 may be configured to determine information related to positions of the plurality of EIT electrodes in the first image. Resolution of a reconstructed EIT image would be improved if the positions of the plurality of EIT electrodes were known beforehand. As the patient may lose or gain weight during the radiotherapy period, or the patient may be positioned slightly different at different times of radiotherapy, it is difficult to reproduce the exact electrode positions at different times. By determining information related to positions of the plurality of EIT electrodes in the first image before the treatment, the EIT images may be reconstructed with the known positions of the plurality of EIT electrodes, and therefore, improving the EIT image quality.

The EIT image generation unit 640 may generate an EIT image of the patient based on the first image and the information related to the plurality EIT electrodes. In some embodiments, the EIT image may be generated according to the acquired electrical impedance data. In some embodiments, the EIT image may be generated according to the information contained in the first image, and information related to the plurality of EIT electrodes.

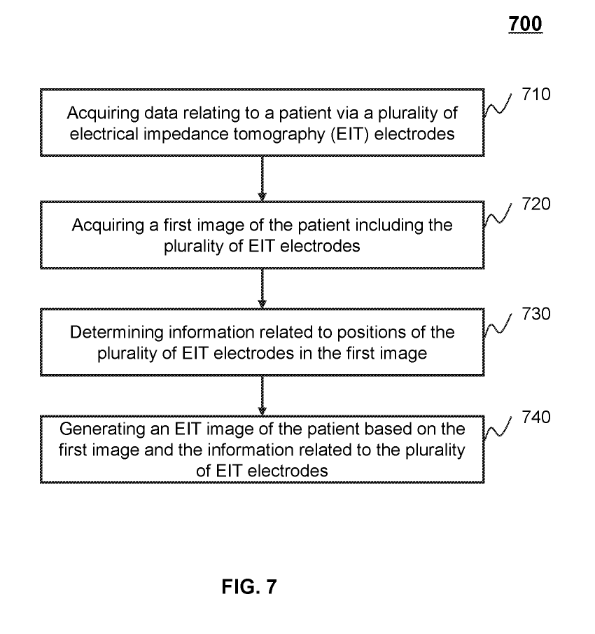

FIG. 7 is a flowchart illustrating an exemplary process/method 700 for generating an EIT image according to some embodiments of the present disclosure. The process and/or method 700 may be executed by the medical system 100. For example, the process and/or method 700 may be implemented as a set of instructions (e.g., an application) stored in the storage 220. The processor 210 may execute the set of instructions and may accordingly be directed to perform the process and/or method 700. The operations of the illustrated process/method presented below are intended to be illustrative. In some embodiments, the process/method may be accomplished with one or more additional operations not described, and/or without one or more of the operations discussed. Additionally, the order in which the operations of the process/method as illustrated in FIG. 7 and described below is not intended to be limiting.

In step 710, the data acquisition unit 610 may acquire data relating to a patient via a plurality of electrical impedance tomography (EIT) electrodes. The plurality of EIT electrodes may be connected to the patient's body. For example, the plurality of EIT electrodes may be placed on the skin of the patient's body. As another example, the plurality of EIT electrodes may be placed within body cavities or orifices of the patient. In some embodiments, the data may include voltage data, current data, or the like, or any combination thereof. The current data may be determined by the exciting system 170, and the current may act on the patient's body via the plurality of EIT electrodes. The voltage data may be determined by detecting the voltage relating to the patient's body (e.g., the head) via the plurality of EIT electrodes. The electrical impedance data may be determined according to the current data, the voltage data, etc.

In step 720, the image acquisition unit 620 may acquire a first image of the patient including the plurality of EIT electrodes. In some embodiments, the image acquisition unit 620 may acquire the first image from the storage 220 or other storage device. The first image of the patient may be obtained to include information related to the plurality of EIT electrodes by imaging a body part of the patient and the plurality of EIT electrodes simultaneously. In some embodiments, the first image may include information relating to electrical characteristics of the plurality of EIT electrodes. The electrical characteristics of the plurality of EIT electrodes may be determined from the first image. In some embodiments, the electrical characteristics of the plurality of EIT electrodes may be used to reconstruct the EIT image, and resolution of a reconstructed EIT image may be improved accordingly.

In step 730, the position determination unit 630 may determine information related to positions of the plurality of EIT electrodes in the first image. Resolution of a reconstructed EIT image would be improved if the positions of the plurality of EIT electrodes were known beforehand. As the patient may lose or gain weight during the radiotherapy period, or the patient may be positioned slightly different at different times of radiotherapy, it is difficult to reproduce the exact electrode positions at different times. Tattoos on the skin are routinely used during radiotherapy as position references. However, creating a tattoo for each EIT electrode is inconvenient. Positions of the tattoos on the skin with respect to internal anatomical structures may also be subject to shift when a patient gains or loses body mass. By determining information related to positions of the plurality of EIT electrodes in the first image before the treatment, the EIT images may be reconstructed with the known positions of the plurality of EIT electrodes, and therefore, improving the EIT image quality.

In step 740, the EIT image generation unit 640 may generate an EIT image of the patient based on the first image and the information related to the plurality of EIT electrodes. In some embodiments, the EIT image may be generated according to the acquired electrical impedance data. In some embodiments, the EIT image may be reconstructed according to the information contained in the first image, and information related to the plurality of EIT electrodes. The quality of the EIT image may be improved accordingly.

It should be noted that the above description of the process/method for generating the EIT image is provided for the purpose of illustration, and is not intended to limit the scope of the present disclosure. For persons having ordinary skills in the art, multiple variations and modifications may be made under the teaching of the present disclosure. However, those variations and modifications do not depart from the scope of the present disclosure. In some embodiments, a correction step may be added to correct the EIT image.

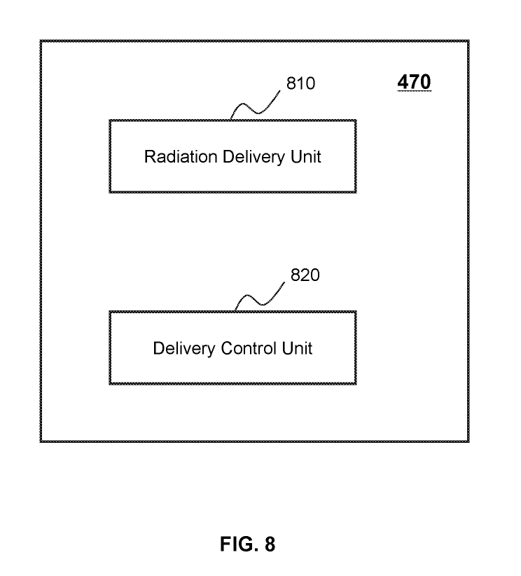

FIG. 8 is a schematic diagram illustrating an exemplary treatment module 470 according to some embodiments of the present disclosure. The treatment module 470 may include a radiation delivery unit 810, and a delivery control unit 820.

The radiation delivery unit 810 may be configured to deliver radiation to an anatomical structure of interest (ASI) of a patient. In some embodiments, the radiation deliver unit 810 may determine the delivery of the radiation to the ASI according a predetermined treatment plan. The predetermined treatment plan may include a radiation dose, a radiation time, or the like, or any combination thereof.

The delivery control unit 820 may be configured to suspend and/or resume the delivery of the radiation to the ASI. For example, if a change in the position relationship between the EIT image and the first image exceeds a pre-set threshold, the delivery control unit 820 may suspend the delivery of the radiation, and spare the OAR. In some embodiment, the delivery control unit 820 may monitor the change of the position relationship between the EIT image and the first image by periodically imaging the patient during the radiotherapy. The periodically imaging may be performed by a planar x-ray system, such as a stereo planar x-ray image pair system.

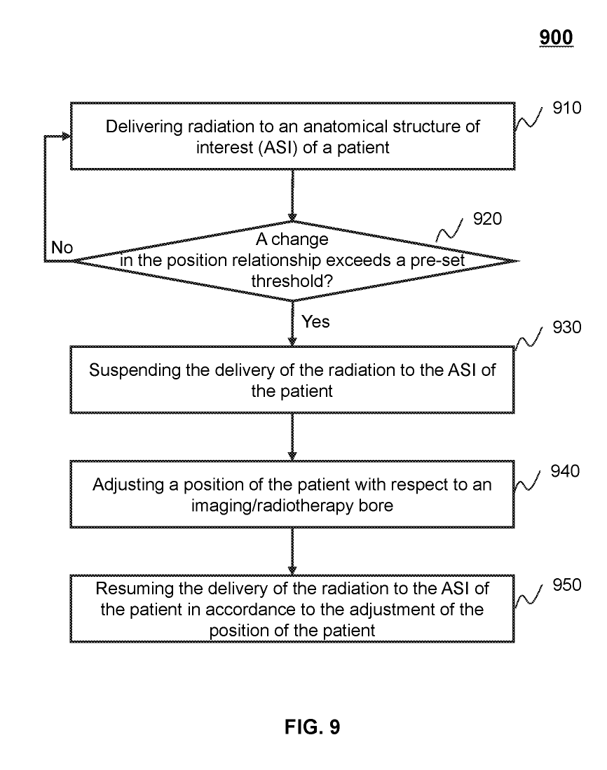

FIG. 9 is a flowchart illustrating an exemplary process/method 900 for controlling radiation delivery according to some embodiments of the present disclosure. The process and/or method 900 may be executed by the medical system 100. For example, the process and/or method 900 may be implemented as a set of instructions (e.g., an application) stored in the storage 220. The processor 210 may execute the set of instructions and may accordingly be directed to perform the process and/or method 900. The operations of the illustrated process/method presented below are intended to be illustrative. In some embodiments, the process/method may be accomplished with one or more additional operations not described, and/or without one or more of the operations discussed. Additionally, the order in which the operations of the process/method as illustrated in FIG. 9 and described below is not intended to be limiting.

In step 910, the treatment module 470 (e.g., the radiation delivery unit 810) may deliver radiation to an anatomical structure of interest (ASI) of a patient. In some embodiments, the radiation delivery unit 810 may deliver radiation to the ASI of the patient according to a predetermined treatment plan. The predetermined treatment plan may include a radiation dose, a radiation time, or the like, or any combination thereof. For example, the radiation delivery unit 810 may start the delivery of the radiation to the ASI when the position of the ASI is conformed to a predetermined treatment plan. As another example, the radiation delivery unit 810 may determine the delivery of the radiation to the ASI by applying a treatment plan that conforms to the position of the ASI.

In step 920, the relationship determination module 440 may determine whether a change in the position relationship exceeds a pre-set threshold. In some embodiments, the position relationship between the EIT image and the first image may be changed during the radiotherapy. The change may be caused by the movement of the patient. The pre-set threshold may be set according to different organs and body parts. In responding to the determination that the change exceeds a pre-set threshold, the process may proceed to step 930. In responding to the determination that the change does not exceed a pre-set threshold, the process may proceed to step 910, and the delivery of the radiation to the ASI continues. In some embodiments, the delivery control unit 820 may monitor the change of the position relationship between the EIT image and the first image by periodically imaging the patient during the radiotherapy. The periodically imaging may be performed by a planar x-ray system, such as a stereo planar x-ray image pair system.

In step 930, the treatment module 470 (e.g., the delivery control unit 820) may suspend the delivery of the radiation to the ASI of the patient if it is determined that the position relationship exceeds a pre-set threshold.

In step 940, the position adjustment module 480 may adjust a position of the patient with respect to an imaging/radiotherapy bore. In some embodiments, the position of the patient may be determined by referring to the position of an ASI of the patient. According to the present disclosure, the patient may be scanned or receive radiation through the radiotherapy bore. In some embodiments, the position of the patient may be adjusted to align with the radiotherapy bore by moving the subject couch 116. In some embodiments, the position adjustment module 480 may also adjust the radiation area relative to the patient. For example, the position adjustment module 480 may adjust the position of the radiation source relative to the patient, so that the treatment module 470 may deliver the radiation to the ASI and spare the organs-at-risk (OAR). In another example, the position adjustment module 480 may adjust the position of the patient relative to the radiation source, so that the treatment module 470 may deliver the radiation to the ASI and spare the organs-at-risk (OAR). In another example, the position adjustment module 480 may adjust the collimators of the radiation source, so that the treatment module 470 may deliver the radiation to the ASI and spare the organs-at-risk (OAR).

In step 950, the treatment module 470 (e.g., the delivery control unit 820) may resume the delivery of the radiation to the ASI of the patient in accordance to the adjustment of the position of the patient. The position relationship between the EIT image and the first image may be updated simultaneously in accordance to the adjustment of the position of the patient. Thus, the ASI of the patient may be re-located and/or tracked according to the position relationship and the motion of the EIT feature. The delivery of the radiation to the ASI may be resumed accordingly.

It should be noted that the above description of the process/method for controlling radiation delivery is provided for the purpose of illustration, and is not intended to limit the scope of the present disclosure. For persons having ordinary skills in the art, multiple variations and modifications may be made under the teaching of the present disclosure. However, those variations and modifications do not depart from the scope of the present disclosure.

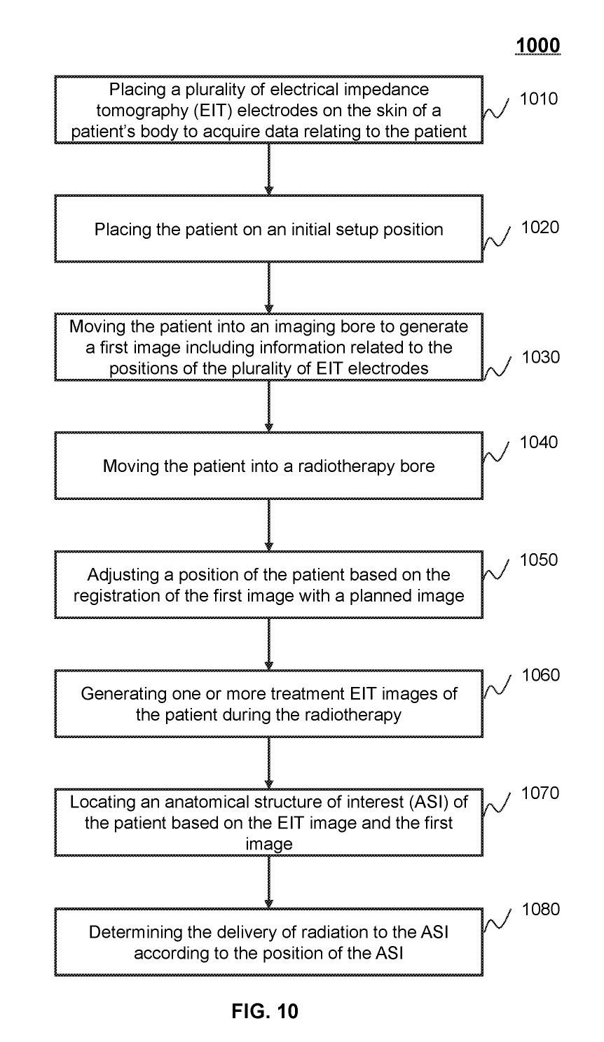

FIG. 10 is a flowchart illustrating an exemplary process/method 1000 for performing a radiotherapy operation by using the medical system 100 according to some embodiments of the present disclosure. The process and/or method 1000 may be executed by the medical system 100. For example, the process and/or method 1100 may be implemented as a set of instructions (e.g., an application) stored in the storage 220. The processor 210 may execute the set of instructions and may accordingly be directed to perform the process and/or method 1000. The operations of the illustrated process/method presented below are intended to be illustrative. In some embodiments, the process/method may be accomplished with one or more additional operations not described, and/or without one or more of the operations discussed. Additionally, the order in which the operations of the process/method as illustrated in FIG. 10 and described below is not intended to be limiting.

In step 1010, a plurality of electrical impedance tomography (EIT) electrodes may be placed on the skin of a patient's body to acquire data relating to the patient. For example, the plurality of EIT electrodes may be placed on the skin of the head, the skin of the neck, the skin of the wrist, the skin of the chest, the skin of the abdomen, etc. In some embodiments, the plurality of EIT electrodes may be placed within the body cavities or orifices of the patient. In some embodiments, the acquired data may include information relating to a bone of the patient, a tissue of the patient, an organ of the patient, etc. The acquired data may be used to generate an EIT image. Before the radiotherapy, the scanned EIT image may be generated based on the acquired data.

In step 1020, the patient may be placed on an initial setup position. In some embodiments, one or more tattoos on the skin of the patient are aligned with one or more room lasers. In some embodiments, the patient may be placed on the subject couch 116. The patient may be placed on the initial setup position by moving the subject couch 116. The initial setup position may be an isocenter of a medical machine, e.g., the imaging machine 112, or the treatment machine 114. The one or more tattoos on the skin of the patient may be marked during a planned imaging process. The planned imaging process may generate a planned image, and a predetermined treatment plan may be determined according to the planned image.

In step 1030, the patient may be moved into an imaging bore to generate a first image. In some embodiments, the first image may include information related to the positions of the plurality of EIT electrodes. Through the imaging bore, the patient may be scanned. The first image may be obtained to include information related to the positions of the plurality of EIT electrodes by imaging a body part of the patient and the plurality of EIT electrodes simultaneously.

In step 1040, the patient may be moved into a radiotherapy bore. Through the radiotherapy bore, the radiation may be delivered to the patient. In some embodiments, the imaging machine 112 and the treatment machine 114 may have collinear bores. The patient may be moved from the imaging bore to the radiotherapy bore by transporting the subject couch 116 along a single axis with the plurality of EIT electrodes placed on the skin of a patient's body or placed within the body cavities or orifices of the patient. In some embodiments, the imaging machine 112 and the treatment machine 114 may have collinear rotation axis. In some embodiments, the imaging machine 112 and the treatment machine 114 may be integrated to a medical device. For example, the imaging machine 112 and the treatment machine 114 may share a same radiation source. In this case, an additional radiation source may be mounted on the gantry. Thus, the imaging bore and the radiotherapy bore may be the same bore of the medical device. And step 1040 will be omitted.

In step 1050, a position of the patient may be adjusted based on the registration of the first image with the planned image. The radiotherapy treatment plan may be generated based on the planned image. The location of an anatomical structure of interest (ASI) in the patient may be changed since the patient may gain or lose weight during the period before the radiotherapy and after the planned image is taken. The change of the location of the ASI in the patient may be determined according to registration of the first image with the planned image. The position of the patient may be adjusted with respect to the imaging/radiotherapy bore according to the change of the position of the ASI in the patient.

In step 1060, one or more treatment EIT images of the patient may be generated during the radiotherapy. In some embodiment, the one or more treatment EIT images of the patient may be generated based on the first image and information related to the plurality of EIT electrodes. In some embodiments, the one or more treatment EIT images may be generated according to the acquired electrical impedance data. In some embodiments, the treatment EIT image may be reconstructed according to the information contained in the first image, the information related to the plurality of EIT electrodes, as well as the acquired electrical impedance data. As the plurality of EIT electrodes are placed on the skin of the patient's body during the radiotherapy process, the treatment EIT image may be obtained continuously. The treatment EIT image may include at least one EIT feature indicating observable structures in the treatment EIT image. The word “observable” may refer to that the structure or the feature can be seen by naked eyes of human beings in the EIT image (e.g., larger than 0.55 mm in the EIT image). The motion of the EIT feature in the treatment EIT image may be tracked simultaneously according to the continuously obtained treatment EIT image.

In step 1070, an anatomical structure of interest (ASI) of the patient may be located based on the EIT image and the first image. A relationship between the EIT image and the first image may be determined according to the EIT feature and the ASI in the first image. In some embodiments, the EIT feature of the EIT image may serve as a suitable surrogate for the ASI. The ASI may be located according to the EIT image and the first image. Besides, the motion of the ASI may be determined according to the motion of the EIT feature.

In step 1080, the delivery of radiation to the ASI may be determined according to the position of the ASI. In some embodiments, the delivery of the radiation to the ASI may be started when the position of the ASI is conformed to the predetermined treatment plan. In some embodiments, the delivery of the radiation to the ASI may be determined by applying a treatment plan that conforms to the position of the ASI. For example, the position of the radiation source relative to the patient may be adjusted, so that the delivery of the radiation to the ASI is determined. In another example, the position adjustment module 480 may adjust the position of the patient relative to the radiation source, so that the treatment module 470 may deliver the radiation to the ASI and spare the organs-at-risk (OAR). In another example, the position adjustment module 480 may adjust the collimators of the radiation source, so that the treatment module 470 may deliver the radiation to the ASI and spare the organs-at-risk (OAR).

In some embodiments, the delivery of the radiation to the ASI may be suspended if a change in the position relationship between the EIT image and the first image exceeds a pre-set threshold.

It should be noted that the above description of the process/method for performing a radiotherapy operation is provided for the purpose of illustration, and is not intended to limit the scope of the present disclosure. For persons having ordinary skills in the art, multiple variations and modifications may be made under the teaching of the present disclosure. However, those variations and modifications do not depart from the scope of the present disclosure. In some embodiments, an imaging step may be added during the radiotherapy process to generate a new image. The new image may be used to determine whether the position relationship between the EIT image and the new image has changed.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

A non-transitory computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB. NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities, properties, and so forth, used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

We claim:

1. A radiotherapy system implemented on one machine, including at least one processor and a storage, the system comprising:
an electrical impedance tomography (EIT) module configured to generate an EIT image of a patient;
a first imaging module configured to generate a first image of the patient;
a feature determination module configured to determine an EIT feature of the EIT image;
a relationship determination module configured to determine a position relationship between the EIT image and the first image;
an anatomical structure of interest (ASI) determination module configured to locate an anatomical structure of interest (ASI) of the patient based on the position relationship; and
a treatment module configured to deliver radiation to the ASI of the patient.

2. The system of claim 1, wherein the EIT image of the patient is generated by a plurality of EIT electrodes connected to the patient's body.

3. The system of claim 2, wherein the first image of the patient includes the plurality of EIT electrodes.

4. The system of claim 3, wherein the EIT module is further configured to:
reconstruct the EIT image of the patient based on the plurality of EIT electrodes and information contained in the first image.

5. The system of claim 3, wherein the EIT module is further configured to:
generate the EIT image of the patient based on information related to positions of the plurality of EIT electrodes obtained from the first image.

6. The system of claim 1, wherein the EIT feature indicates an observable structure of the EIT image.

7. The system of claim 1, wherein the EIT image further include a scanned EIT image and a treatment EIT image.

8. The system of claim 7, wherein the relationship determination module is further configured to:
generate the position relationship between the EIT image and the first image based on the EIT feature of the scanned EIT image and the ASI in the first image.

9. The system of claim 1, wherein
the relationship determination module is further configured to determine a change in the position relationship during the delivery of the radiation to the ASI of the patient,
the treatment module is further configured to suspend the delivery of the radiation in response to a determination that the change in the position relationship exceeds a pre-set threshold,
the system further comprises a position adjustment module configured to adjust the position of the patient relative to a radiation source; and
the treatment module is further configured to resume the delivery of the radiation to the ASI of the patient in accordance to the adjustment of the position of the patient.

10. A radiotherapy method implemented on one machine, including at least one processor and a storage, the method comprising:
generating an electrical impedance tomography (EIT) image of a patient;
generating a first image of the patient;
determining an EIT feature of the EIT image;
determining a position relationship between the EIT image and the first image;
locating an anatomical structure of interest (ASI) of the patient based on the position relationship; and
delivering radiation to the ASI of the patient.

11. The method of claim 10, wherein the EIT image of the patient is generated by a plurality of EIT electrodes connected to the patient's body.

12. The method of claim 11, wherein the first image of the patient includes the plurality of EIT electrodes.

13. The method of claim 12, further comprising:
reconstructing the EIT image of the patient based on the plurality of EIT electrodes and information contained in the first image.

14. The method of claim 12, further comprising:
generating the EIT image of the patient based on information related to positions of the plurality of EIT electrodes obtained from the first image.

15. The method of claim 10, wherein the EIT feature indicates an observable structure of the EIT image.

16. The method of claim 10, wherein the EIT image further include a scanned EIT image and a treatment EIT image.

17. The method of claim 16, further comprising:
generating the position relationship between the EIT image and the first image based on the EIT feature of the scanned EIT image and the ASI in the first image.

18. The method of claim 10, further comprising:
determining a change in the position relationship during the delivery of the radiation to the ASI of the patient;
suspending the delivery of the radiation in response to a determination that the change in the position relationship exceeds a pre-set threshold;
adjusting the position of the patient relative to a radiation source; and
resuming the delivery of the radiation to the ASI of the patient in accordance to the adjustment of the position of the patient.

19. A method for radiotherapy, comprising:
generating a scanned EIT image of a patient;
generating a first image of the patient at a first position;
determining an EIT feature of the scanned EIT image;
determining an anatomical structure of interest (ASI) in the first image;
determining a position relationship between the EIT feature and the ASI based on the scanned EIT image and the first image;
moving the patient from the first position to a second position;
generating a treatment EIT image of the patient at the second position;
identifying the EIT feature on the treatment EIT image;
determining a position of the ASI based on the EIT feature and the position relationship between the EIT feature and the ASI; and
delivering radiation to the ASI based on the position of the ASI.

20. The method of claim 19, wherein the first position is an imaging bore of an imaging machine, and the second position is a radiotherapy bore of a treatment machine.

21. The method of claim 20, wherein the imaging machine and the treatment machine have collinear bores.

22. The method of claim 20, wherein the imaging machine and the treatment machine have collinear rotation axis.

23. The method of claim 19, wherein a plurality of EIT electrodes are connected to the patient's body during generating the first image, during moving the patient from the first position to the second position, and during delivering the radiation to the ASI based on the position of the ASI.
